(19) United States
(12) Patent Application Publication
(10) Pub. No.: US 2014/0127232 A1
(43) Pub. Date: May 8, 2014

(54) DETECTING, PREVENTING, AND TREATING BRONCHOPULMONARY DYSPLASIA

(71) Applicants: Thomas J. Mariani, Pittsford, NY (US); Gloria S. Pryhuber, Rochester, NY (US)

(72) Inventors: Thomas J. Mariani, Pittsford, NY (US); Gloria S. Pryhuber, Rochester, NY (US)

(73) Assignee: UNIVERSITY OF ROCHESTER, Rochester, NY (US)

(21) Appl. No.: 14/118,042

(22) PCT Filed: May 16, 2012
(86) PCT No.: PCT/US2012/038090

§ 371 Date: Jan. 3, 2014

Related U.S. Application Data
(60) Provisional application No. 61/486,613, filed on May 16, 2011.

(51) Int. Cl. G01N 33/573

(52) U.S. Cl. CPC G01N 33/573
USPC 424/158.1; 435/6.11; 435/6.12; 435/6.18; 435/7.4; 506/9; 506/18; 514/1.5; 514/44 R; 514/44 A

(57) ABSTRACT

Provided herein are methods for preventing or treating bronchopulmonary dysplasia (BPD) in a subject. The methods comprise identifying connective tissue mast cells (CTMCs) in a subject and administering an agent that blocks an activity of blocks an increase in a level of or reduces a level of the CTMCs in the subject. Also provided are methods of identifying a subject with or at risk for developing BPD. Also provided are methods for determining the effectiveness of a treatment regime for BPD in a subject.

### CROSS-REFERENCE TO PRIORITY APPLICATION

This application claims priority to U.S. Provisional Application No. 61/486,613, filed May 16, 2011, which is incorporated herein by reference in its entirety.

### BACKGROUND

Bronchopulmonary Dysplasia (BPD) is a lung disease of the premature infant defined by dependence on supplemental oxygen for more than 28 days post-partum and/or at 36 weeks corrected gestational age. In the United States, more than 500,000 babies are born prematurely each year, with approximately 60,000 at high risk for BPD and 10,000 diagnosed with this disease (Chronic Lung Disease after Premature Birth Eugenio Baraldi, M.D., and Marco Filippone, M.D. N Engl J Med 2007; 357:1946-1955). The overall costs of treating infants with BPD in the United States are estimated to be $2.5 billion, second only for pediatric lung disease to the costs for treating asthma and far exceeding the cost of treating cystic fibrosis. The incidence of BPD has increased over the last 30 years, largely due to higher survival rates of premature babies.

### SUMMARY

Provided herein are methods for preventing or treating bronchopulmonary dysplasia (BPD) in a subject. The methods comprise directly or indirectly identifying a subset of mast cells, chymase-expressing connective-tissue mast cells (CTMCs), in a biological sample from the subject and administering to the subject an agent that blocks an activity of, blocks an increase in, reduces activation of, or reduces the level of chymase-expressing CTMCs in the subject.

Also provided are methods of identifying a subject with or at risk of developing BPD. The methods comprise directly or indirectly detecting chymase-expressing CTMCs in a biological sample from the subject. The presence of chymase-expressing CTMCs, as compared to a control, indicates the subject has BPD.

Also provided are methods of determining the effectiveness of a treatment regime for BPD in a subject. The methods comprise identifying a first level of chymase-expressing CTMCs, directly or indirectly, in a first biological sample from the subject before administration of the treatment regime, identifying a second level of chymase-expressing CTMCs in a second biological sample from the subject after administration of the treatment regime, and adjusting the treatment regime if the second level of chymase-expressing CTMCs is the same or higher than the first level. The method optionally comprises a third or subsequent identification step or steps.

Further provided are assay systems comprising, for example, a microarray or a gene chip with at least two selective binding agents. Each selective binding agent is specific for a biomarker selected from the group consisting of chymase and/or carboxypeptidase A3 (CPA3), optionally with a binding agent specific for tryptase beta 2 (TPSB2), tryptase alpha/beta 1 (TPSAB1), cathepsin G, and/or prostaglandin D2 (PGD2).

The details of one or more embodiments are set forth in the accompanying drawings and the description below. Other features, objects, and advantages will be apparent from the description and drawings, and from the claims.

### DESCRIPTION OF DRAWINGS

FIG. 1 shows canonical pathways affected in bronchopulmonary dysplasia (BPD) lungs. In order to estimate general biological mechanisms that are altered during BPD pathogenesis, pathways analysis using Ingenuity software (Ingenuity Systems; Redwood City, Calif.) was performed. 159 genes whose expression was significantly altered in BPD lung tissue were identified and over-representation of this gene set was assessed in known pathways. Shown are “canonical” pathways, ranked by Fisher's-exact T-test p-value, listing the percentage of the BPD-associated gene set in each pathway (dark gray bar), versus the percentage of all genes in that pathway (light gray bar).

FIG. 2 shows a graph demonstrating validation of gene expression. Quantitative real-time PCR (qPCR) was used to confirm expression patterns predicted by microarray analysis. Nine genes were selected based upon their magnitude of change and biological interest. Shown are fold-differences in expression (log scale) for each gene, comparing all samples, or samples divided by gestational age. All genes demonstrated evidence for replication, with 7 of 9 displaying significant differences between BPD and controls.

FIG. 3 shows a graph showing increased mast cell marker expression in an animal model of BPD using qPCR to assess gene expression for the mast cell markers CPA3, TPSAB1 and TPDB2 in whole lung tissue obtained from wild type (WT, n≧3) orFGFR3/4 compound deficient (KO, n≧3) mice at 1 month of age. Shown are mean relative gene expression values for each group and for each gene (*, p-value<0.05).

FIG. 4 shows an increased mast cell accumulation in BPD lungs. Immunohistochemistry was used to identify tryptase-expressing cells, a general mast cell-specific marker. FIG. 4A (top left) shows quantitative analysis of the number of tryptase-expressing cells in BPD and non-BPD control lungs. Shown are the average numbers of stained cells/field for each individual subject (dots), the group means (bar) and interquartile range (box). FIGS. 4B and 4C (top and middle right) show examples of staining patterns in age-matched control subjects. Control staining is shown as Non-Immune Serum. FIGS. 4D and 4E (bottom left and right) are bar graphs showing examples of staining patterns in BPD subjects. Distribution of stained cells within the alveolar, peribronchiolar or perivascular region of BPD and control tissues. Data are presented as the average absolute number of cells per subject in each region (bottom left) or the frequency (proportion) of stained cells found in each region (bottom right). Magnification is as indicated on scale bars. A significant approximate 5-fold increase in tryptase positive cells was observed in BPD lungs.

FIG. 5 shows an increased connective tissue-type mast cell accumulation in BPD lungs. Immunohistochemistry was used to identify chymase-expressing cells, a specific marker for connective tissue-type mast cells. Quantitative analysis of the number of chymase-expressing cells in BPD and non-BPD control lungs is indicated at the top left (5A). Shown are the average numbers of stained cells/field for each individual subject (dots), the group means (bar) and interquartile range (box). FIGS. 5B-5C (top and middle right) show individual images of staining patterns in corrected gestational age matched control and BPD subjects (subject ID indicated). Panels are arranged according to gestational age at death. Also shown is an absence of staining in non-immune serum controls. Bar graphs at bottom show distribution of stained cells within the alveolar, peribronchiolar or perivascular region of BPD and control tissues. Data are presented as the average absolute number of cells per subject in each region (bottom left) or the frequency (proportion) of stained cells found in each region (bottom right). Bar graphs at bottom show distribution of stained cells within the alveolar, peribronchiolar or perivascular region of BPD and control tissues. Data are presented as the average absolute number of cells in each region (bottom left) or the frequency of stained cells found in each region (bottom right).

FIG. 6 shows the amount of chymase detected in infants relative to the day after birth on which the sample was collected. Serial tracheal aspirates were collected from a set of quadruplets born at less than 29 weeks. Chymase was detected by ELISA in each sample and is presented relative to the day after birth on which the sample was collected. Unique patterns of tracheal aspirate chymase content were detected in each infant. FIG. 6A shows the amount of chymase detected in subject 48 relative to the day after birth on which the sample was collected. FIG. 6B shows the amount of chymase detected in subject 49 relative to the day after birth on which the sample was collected. FIG. 6C shows the amount of chymase detected in subject 50 relative to the day after birth on which the sample was collected. FIG. 6D shows the amount of chymase detected in subject 51 relative to the day after birth on which the sample was collected.

### DETAILED DESCRIPTION

Bronchopulmonary dysplasia (BPD) is a major complication of premature birth. Thus, premature infants, particularly infants born at less than 32 weeks of gestation or weighing less than 1500 grams (about 3.3 pounds) are at a risk for developing BPD. In addition to weight and gestational age at birth, risk factors for BPD are complex and include prenatal or perinatal infection, O₂ toxicity, and ventilation-associated injury. Thus, infection, O₂ administration, and ventilators also indicate an infant at risk for BPD. BPD pathology is complex and characterized by inflammation, dysmorphic airspaces and vasculature, and aberrant extracellular matrix accumulation.

Provided herein are methods for preventing or treating bronchopulmonary dysplasia (BPD) in a subject. The methods comprise identifying connective tissue mast cells (CTMCs) in a biological sample from the subject and administering to the subject an agent that blocks an activity of, blocks an increase in a level of, or reduces a level of CTMCs in the subject. The CTMCs are a subset of mast cells. They can be positive for such biomarkers as chymase, carboxypeptidase A3 (CPA3), tryptase beta 2 (TBSP2), tryptase alpha/beta 1 (TBSAB 1), cathepsin G (CTSG), and prostaglandin D2 (PGD2). Blocking an activity of, blocking an increase in a level of, or reducing a level of CTMCs results in the prevention or treatment of BPD in the subject.

Blocking an activity of a CTMC (e.g., a chymase-expressing CTMC) can, for example, include blocking the activation of the CTMC. Blocking the activation of a CTMC can, for example, result in blocking the release of granules (e.g., histamine granules) and various hormonal mediators into the interstitium. Blocking activation of CTMCs reduces the inflammatory process. Blocking an increase in the level of or reducing a level of a CTMC as used herein, means the number of CTMCs stays the same or decreases as compared to a control (e.g., a known number or level or a control without BPD).

As used herein a control can be a treated or untreated, non-BPD diseased sample from a different subject. Optionally, a control can be an untreated sample from the same subject. Optionally, a control can be a reference value previously obtained for a treated or untreated, non-BPD diseased sample or for a treated or untreated, diseased sample. In the case of a non-BPD diseased sample, as described herein, there are no CTMCs or a very low level of CTMCs; however, there is a baseline level of tryptase-expressing mast cells (MC_{T}).

The CTMCs can, for example, express an increased level of chymase protein or mRNA and/or an increased level of carboxypeptidase A3 (CPA3) protein or mRNA as compared to a non-connective tissue mast cell. A non-connective tissue mast cell can, for example, include any type of mast cell that is not a connective tissue mast cell. A non-connective tissue mast cell is positive for tryptase or other general mast cell biomarkers, but is negative for connective-tissue mast cell biomarkers such as chymase.

Optionally, the agent used to treat or prevent BPD blocks expression or activity of CPA3 or chymase. Blocking expression of CPA3 or chymase, as used herein, means the agent decreases the level of expression or inhibits expression of CPA3 or chymase. Blocking the activity of CPA3 or chymase, as used herein, means that CPA3 or chymase cannot perform their natural protease function.

The agent can, for example, be a neutralizing antibody to CPA3 or chymase. The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

Functional antibody fragments or single chain antibodies can also be used. The term antibody or fragments thereof can also encompass chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain CPA3 or chymase binding activity are included within the meaning of the term antibody or fragment thereof Such antibodies and fragments can be made by techniques known in the art and can be screened for specificity and activity according to general methods for producing antibodies and screening antibodies for specificity and activity (See Harlow and Lane. Antibodies, A Laboratory Manual. Cold Spring Harbor Publications, New York (1988)).

Also included within the meaning of antibody or fragments thereof are conjugates of antibody fragments and antigen binding proteins (single chain antibodies) as described, for example, in U.S. Pat. No. 4,704,692, the contents of which are hereby incorporated by reference in their entirety.

Optionally, the CTMCs express an increased level of one or more biomarkers selected from the group consisting of chymase, carboxypeptidase A3 (CPA3), tryptase beta 2 (TBSP2), tryptase alpha/beta 1 (TBSAB1), cathepsin G (CTSG), and prostaglandin D2 (PGD2).

Optionally, the agent can be selected from the group consisting of a small molecule, a polypeptide, a nucleic acid, or a peptidomimetic. The nucleic acid can, for example, be selected from the group consisting of a small interfering RNA (siRNA), a microRNA (miRNA), or an antisense nucleic acid.

As used herein, an inhibitory nucleic acid sequence can be a short-interfering RNA (siRNA) sequence or a micro-RNA (miRNA) sequence that is specific for the mRNA of one or more of the biomarkers (e.g., carboxypeptidase A3, chymase, tryptase beta 2 (TPSB2), and tryptase alpha/beta 1 (TPSAB1)). A 21-25 nucleotide siRNA or miRNA sequence can, for example, be produced from an expression vector by transcription of a short-hairpin RNA (shRNA) sequence, a 60-80 nucleotide precursor sequence, which is subsequently processed by the cellular RNAi machinery to produce either a siRNA or miRNA sequence. Alternatively, a 21-25 nucleotide siRNA or miRNA sequence can, for example, be synthesized chemically. Chemical synthesis of siRNA or miRNA seuquences is commercially available from such corporations as Dharmacon, Inc. (Lafayette, Colo.), Qiagen (Valencia, Calif.), and Ambion (Austin, Tex.). A siRNA sequence preferably binds a unique sequence within the mRNA with exact complementarity and results in the degradation of the mRNA molecule. A siRNA sequence can bind anywhere within the mRNA molecule. A miRNA sequence preferably binds a unique sequence within the mRNA with exact or less than exact complementarity and results in the translational repression of the mRNA molecule. A miRNA sequence can bind anywhere within the mRNA sequence, but preferably binds within the 3′ untranslated region of the mRNA molecule. Methods of delivering siRNA or miRNA molecules are known in the art. See, e.g., Oh and Park, Adv. Drug. Deliv. Rev. 61(10):850-62 (2009); Gondi and Rao, J. Cell Physiol. 220(2):285-91 (2009); and Whitehead et al., Nat. Rev. Drug. Discov. 8(2):129-38 (2009).

As used herein, an inhibitory nucleic acid sequence can be an antisense nucleic acid sequence. Antisense nucleic acid sequences can, for example, be transcribed from an expression vector to produce an RNA which is complementary to at least a unique portion of the biomarker mRNA and/or the endogenous gene which encodes the biomarker. Hybridization of an antisense nucleic acid under specific cellular conditions results in inhibition of the biomarker protein expression by inhibiting transcription and/or translation.

Also provided are methods for determining the effectiveness of a treatment regime for BPD in a subject. The methods comprise identifying a first level of CTMCs in a first biological sample from the subject before administration of a treatment regime to the subject, identifying a second level of CTMCs in a second biological sample from the subject after administration of a treatment regime to the subject, comparing the first and second level of CTMCs, and adjusting the treatment regime if the second level of CTMCs is the same or higher than the first level. Optionally, for a treatment regime that blocks the activity of a CTMC (e.g., blocks the activation of the chymase-expressing CTMC), the methods comprise determining a first and second level of activation of the CTMCs, comparing the first and second level of activation, and adjusting the treatment regime if the second level of activation is the same or higher than the first level of activation. The determining, comparing, and adjusting steps can be repeated, as needed, over the course of the treatment regime or over the course of various treatment regimes.

Also provided are methods of identifying a subject with or at risk for developing BPD. The methods comprise detecting CTMCs in a biological sample from the subject, wherein the presence of CTMCs as compared to a control indicates the subject has BPD.

As used herein, a biological sample can include, a sample selected from the group consisting of blood, an airway aspirate, a tracheal aspirate, a bronchoalveolar lavage (BAL), urine, and lung tissue. An airway aspirate can include a lower airway washing (e.g., a deep lung washing) or an upper airway washing (e.g., a nasal washing or nasal scraping). A biological sample can also be a biopsy of lung tissue.

The CTMCs can be detected directly or indirectly. Direct detection can, for example, include histological detection of CTMC in the biological sample. Indirect detection can, for example, include detection of one or more secreted CTMC biomarkers. Thus, optionally, detection of CTMCs comprises detecting expression of one or more biomarkers selected from the group consisting of chymase, CPA3, TPSB2, TPSAB1, CTSG, and PGD2.

The level of expression of the one or more biomarkers is detected by the level of RNA or polypeptide in the biological sample. Optionally, the level of RNA is determined using an assay selected from the group consisting of a microarray analysis, a gene chip, a Northern blot, an in situ hybridization assay, a RT-PCR assay, a one step PCR assay, and a quantitative real time (qRT)-PCR assay. Optionally, the level of polypeptide is determined using an assay selected from the group consisting of a Western blot, an enzyme-immunosorbent assay (ELISA), an enzyme immunoassay (EIA), a radioimmunoassay (RIA), an immunohistochemistry (IHC) assay, and a protein array. The analytical techniques to determine RNA or polypeptide expression are known. See, e.g. Sambrook et al., Molecular Cloning: A Laboratory Manual, 3^{rd} Ed., Cold Spring Harbor Press, Cold Spring Harbor, N.Y. (2001).

Further provided are assay systems comprising a microarray or gene chip with at least two selective binding agents specific for one or more CTMC biomarkers. Each selective binding agent is specific for a biomarker of a chymase-expressing CTMC selected from the group consisting of chymase, CPA3, TPSB2, TPSAB1, CTSG, and PGD2. The assays systems can, for example, be a DNA microarray or gene chip, a RNA microarray or gene chip, or a protein array. Arrays and gene chips are known in the art. See, e.g., Dufva, Methods Mol. Biol. 529:1-22 (2009); Plomin and Schalkwyk, Dev. Sci. 10:19-23 (2007); Kopf and Zharhary, Int. J. Biochem. Cell Biol. 39(7-8):1305-17 (2007); Haab, Curr. Opin. Biotechnol. 17(4):415-21 (2006); U.S. Pat. No. 5,445,934; U.S. Pat. No. 5,800,992; and U.S. Pat. No. 5,807,552. Optionally, the assay system is limited to binding agents specific for CTMCs but may include one or more control markers as well.

Provided herein are compositions containing the provided small molecules, polypeptides (including, e.g., antibodies or antibody fragments), nucleic acid molecules, and/or peptidomimetics and a pharmaceutically acceptable carrier described herein. The herein provided compositions are suitable for administration in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the small molecule, polypeptide, nucleic acid molecule, and/or peptidomimetic, to humans or other subjects.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by instillation via bronchoscopy. Optionally, the composition is administered by oral inhalation, nasal inhalation, or intranasal mucosal administration. Administration of the compositions by inhalant can be through the nose or mouth via delivery by spraying or droplet mechanism, for example, in the form of an aerosol.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, the nucleic acid molecule or polypeptide is administered by a vector comprising the nucleic acid molecule or a nucleic acid sequence encoding the polypeptide. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retorviruses, Cold Spring Harbor Laboratory Press (1997), which is incorporated by reference herein for the vectors and methods of making them. The construction of replication-defective adenoviruses has been described (Berkner et al., J. Virol. 61:1213-20 (1987); Massie et al., Mol. Cell. Biol. 6:2872-83 (1986); Haj-Ahmad et al., J. Virol. 57:267-74 (1986); Davidson et al., J. Virol. 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrane fusion. Methods for making and using DBs are described in, for example, Pepperl-Klindworth et al., Gene Therapy 10:278-84 (2003).

The provided polypeptides can be delivered by tegument aggregates. Methods for making and using tegument aggregates are described in International Publication No. WO 2006/110728.

Non-viral based delivery methods can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus

As used herein, the terms peptide, polypeptide, or protein are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, subject can be a vertebrate, more specifically a mammal (e.g., a human). The term does not denote a particular sex. The subjects are typically newborns. Thus, newborn subjects, whether male or female, are intended to be covered. As used herein, patient or subject may be used interchangeably and can refer to a subject with a disease or disorder (e.g., bronchopulmonary dysplasia). The term patient or subject includes human and veterinary subjects.

A subject at risk of developing a disease or disorder can be born prematurely (e.g., about 10 weeks before the due date), have breathing problems, low birth weight, prolonged O₂ administration, use of a ventilator, and/or have an infection before, during, or shortly after birth. All of these factors place a neonate at risk for BPD. A subject at risk of developing a disease or disorder can be genetically predisposed to the disease or disorder, e.g., have a family history or have a mutation in a gene that causes the disease or disorder, or show early signs or symptoms of the disease or disorder. A subject currently with a disease or disorder has one or more than one symptom of the disease or disorder and may have been diagnosed with the disease or disorder.

The methods and agents as described herein are useful for both prophylactic and therapeutic treatment. For prophylactic use, a therapeutically effective amount of the agents described herein are administered to a subject prior to onset (e.g., before obvious signs of bronchopulmonary dysplasia) or during early onset (e.g., upon initial signs and/or symptoms of bronchopulmonary dysplasia). Prophylactic administration can occur for several days to a week prior to the manifestation of symptoms of bronchopulmonary dysplasia. Prophylactic administration can be used, for example, in the preventative treatment of subjects at risk of developing bronchopulmonary dysplasia (e.g., a subject diagnosed with a genetic predisposition to bronchopulmonary dysplasia). Therapeutic treatment involves administering to a subject a therapeutically effective amount of the agents described herein after diagnosis or development of bronchopulmonary dysplasia.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or more days. Guidance can be found in the literature for appropriate dosages for given classes of pharmaceutical products.

As used herein the terms treatment, treat, or treating refers to a method of reducing or delaying the effects of a disease or condition or symptom of the disease or condition. Thus, for example, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to control levels (e.g., in the absence of treatment). Treatment can also cause a delay in the onset of new symptoms or further progression of existing symptoms. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications to the agents or steps of the methods are discussed, each and every combination and permutation of the agents and method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLE

### Example 1

### Genome Wide Transcriptional Profiling in Brochopulmonary Dysplasia

### Materials and Methods

### Human Tissue Samples and RNA Isolation.

Over the past decade, under an IRB-approved protocol, a unique biorepository of autopsy tissue samples from human premature babies diagnosed with BPD and non-BPD controls was established. These lung samples were harvested within 6 hours and snap frozen in liquid nitrogen. Estimated gestational age was based on obstetrical dating of the last menstrual period and early trimester ultrasound fetal measurements, confirmed by physical assessment at birth. In order to discover novel BPD biomarkers, 28 of these samples were selected for genome-wide expression profiling, including 11 BPD and 9 control (non-BPD) cases, which were matched for gestational age at birth and death, as well as 4 cases with culture-positive, acute overwhelming sepsis and 4 cases of early postnatal cardiovascular collapse secondary to immaturity or necrotizing enterocolitis (Table 1). Histopathological sections from 4 slides with a diagnosis of BPD, obtained at the Cincinnati Children's Hospital Medical Center were used for replication.

TABLE 1
Subject demographics, including age, and pathological diagnosis.

| | | | | |
|---|---|---|---|---|
| | | | GA at Birth | GA at Death |
| Sample ID | Phenotype | Diagnosis | (wks) | (wks) |

Group 1: Controls
Early Gestation

| | | | | |
|---|---|---|---|---|
| 35 | Control | Extreme prematurity, RDS | 23 | 23.9 |
| 52 | Control | HIE, NLD to very mild RDS | 24.5 | 26.1 |
| 53 | Control | RDS, Pulmoary hemorrhage | 24.4 | 25.0 |
| 56 | Control | NLD to very mild RDS; Central Line Event | 26.6 | 28.5 |

Late Gestation

| | | | | |
|---|---|---|---|---|
| 8 | Control | Neuromuscular Abnormality, NLD | 40 | 40.6 |
| 30 | Control | HIE, NLD | 38.5 | 38.6 |
| 46 | Control | HIE, Acute Meconium Aspiration | 40.9 | 41.0 |
| 50 | Control | HIE, NLD | 41 | 41.4 |
| 51 | Control | NLD to very mild RDS, Central Line Event | 32.3 | 32.7 |

Group 2: Bronchopulmonary Dysplasia (BPD)
Early BPD

| | | | | |
|---|---|---|---|---|
| 10 | BPD | BPD | 26 | 27.7 |
| 11 | BPD | BPD, Necrotizing Enterocolitis | 26 | 34.1 |
| 14 | BPD | BPD, Necrotizing Enterocolitis | 26 | 28.4 |
| 17 | BPD | BPD, CMV | 25 | 34.1 |
| 49 | BPD | BPD, Rhinovirus | 24.7 | 31.8 |
| 58 | BPD | BPD, Acute Pneumonia | 25.4 | 34.0 |

Late BPD

| | | | | |
|---|---|---|---|---|
| 18 | BPD | BPD | 27 | 40.7 |
| 19 | BPD | Healed BPD, Recurrent Necrotizing | 28 | 50.6 |
| | | Enterocolitis | | |
| 33 | BPD | BPD, CMV | 29.2 | 43.1 |
| 44 | BPD | BPD | 28 | 45.0 |
| 47 | BPD | BPD, Cor Pulmonale | 29.1 | 46.5 |

Group 3: Non-BPD lung disease
Acute Inflammation, minimal prior lung disease

| | | | | |
|---|---|---|---|---|
| 13 | Others | Chorioamnionitis; Probable Sepsis; RDS | 27 | 27.1 |
| 29 | Others | Chorioamnionitis; RDS; Pulmonary Hemorrhage | 28 | 28.1 |
| 39 | Others | Resolved Mild RDS; Necrotizing Enterocolitis | 32 | 33.1 |
| 40 | Others | Resolved Mild RDS; Necrotizing Enterocolitis | 31.2 | 32.3 |

Severe Sepsis/Pneumonia

| | | | | |
|---|---|---|---|---|
| 3 | Others | HIE; Necrotizing Pneumonia; Probable | 42 | 43.3 |
| | | Aspiration | | |
| 5 | Others | Necrotizing Enterocolitis; E. coli Sepsis | 28 | 33.0 |
| 20 | Others | E coli Sepsis | 31 | 31.6 |
| 24 | Others | Herpes Simplex Viral Sepsis and Pneumonia | 27.7 | 28.4 |

GA: Gestational Age; BPD: Bronchopulmonary Dysplasia; RDS: Respiratory Distress Syndrome; NLD: no lung disease; HIE: Hypoxic Ischemic Encephalopathy

Frozen tissue was homogenized in Trizol regent (Invitrogen; Carlsbad, Calif.), and total RNA was purified using a two step protocol using the Agilent MiniPrep kit (Agilent Technologies; Santa Clara, Calif.) including an on-column DNase I treatment. The quality of purified RNA was assessed by microcapillary electrophoresis using Bio-Rad Experion® (Bio-Rad; Hercules, Calif.). RNA concentration was determined by spectrophotometry using a NanoDrop ND-300 (Thermo Scientific; Wilmington, Del.). Only RNA samples with an RNA concentration>100 ng/μl and a RNA Integrity Number (RIN)>6 were used for microarray analysis.

### Microarray Profiling.

RNA samples were analyzed using the Affymetrix Human Genome GeneChip U133 Plus 2.0 microarray (Affymetrix; Santa Clara, Calif.), containing 54,675 probes corresponding to 19,501 unique NCBI Entrez genes. RNA from individual samples was transcribed into a labeled target and hybridized to an array. The arrays were washed and scanned according to manufacturer's recommendations. Expression values were extracted from .CEL files using Robust Multi-array Average (RMA) as implemented in BioConductor (Fred Hutchinson Cancer Research Center; Seattle, Wash.).

### Data Analysis.

Ingenuity Pathway Analysis (IPA) was used for gene selection and pathway analysis. Significance in gene expression difference between 11 BPD cases and 9 non-BPD controls was defined using multiple criteria of T-Test p<0.05 and Fold Change>2. Genes meeting these criteria were used for further pathway analysis.

Quantitative Reverse Transcriptase-Polymerase Chain Reaction (qPCR).

qPCR was performed on a Stratagene MX3000P using pre-developed commercial or non-commercial assays. Gene expression levels were calculated relative to the measured Ct value of PPIA (peptidyl prolyl isomerase A or cyclophilin A) as an internal, endogenous control, according to the ddCT method. See Table 2.

TABLE 2
Primer sequences and assay information for qPCR validation
performed in this study.

| Gene | Forward Primer | Reverse Primer |
|---|---|---|
| SYBR Assay | | |
| Human | | |
| PPIA | CCCACCGTGTTCTTCGACATT | GGACCCGTATGCTTTAGGATGA |
| | (SEQ ID NO: 1) | (SEQ ID NO: 2) |
| IGF1 | ATGCTCTTCAGTTCGTGTGTG | GGGCTGATACTTCTGGGTCTT |
| | (SEQ ID NO: 3) | (SEQ ID NO: 4) |
| CCL17 | CTTCTCTGCAGCACATCCAC | CTGCCCTGCACAGTTACAAA |
| | (SEQ ID NO: 5) | (SEQ ID NO: 6) |
| CEACAM5 | CCCAGACTCGTCTTACCTTGC | TTGGCGATAAAGAGAACTTGTGT |
| | (SEQ ID NO: 7) | (SEQ ID NO: 8) |
| CEACAM6 | TCCCCCTCAAAGGCCAATTAC | TGGAACGTCCCATTGATAAACC |
| | (SEQ ID NO: 9) | (SEQ ID NO: 10) |
| SLC27A6 | ACTCCGTATGCCATTTCCAA | TTGGAGAACTTTGTCGCTACC |
| | (SEQ ID NO: 11) | (SEQ ID NO: 12) |
| FABP4 | AGCACCATAACCTTAGATGGGG | CGTGGAAGTGACGCCTTTCA |
| | (SEQ ID NO: 13) | (SEQ ID NO: 14) |
| SFN | AGGCCGAACGCTATGAGGA | GGTTTCGCTCTTCGCAGGA |
| | (SEQ ID NO: 15) | (SEQ ID NO: 16) |
| COL8A1 | GCTGCCACCTCAAATTCCTC | CAGGGGCTGGTATTGTGGA |
| | (SEQ ID NO: 17) | (SEQ ID NO: 18) |
| HHIP | CTTTGGCCCTGACGGCTTT | GTAGCACTGAGCCTGTGAAATC |
| | (SEQ ID NO: 19) | (SEQ ID NO: 20) |
| TEK | TGCCACCCTGGTTTTTACGG | TTGGAAGCGATCACACATCTC |
| | (SEQ ID NO: 21) | (SEQ ID NO: 22) |
| CPA3 | GGGTTTGATTGCTACCACTCTT | GCCAAGTCCTTTATGATGTCTGC |
| | (SEQ ID NO: 23) | (SEQ ID NO: 24) |
| TPSB2 | CCGCGACCGATACTGGATG | GATCTGGGCGGTGTAGAACT |
| | (SEQ ID NO: 25) | (SEQ ID NO: 26) |
| TPSAB1 | GTGACGCAAAATACCACCTTGGC | CCATTCACCTTGCACACCAGGG |
| | (SEQ ID NO: 27) | (SEQ ID NO: 28) |
| Mouse | | |
| CPA3 | AATTGCTCCTGTCCACTTTGAC | TCACTAACTCGGAAATCCACAGT |
| | (SEQ ID NO: 29) | (SEQ ID NO: 30) |
| TPSB2 | CTGGCTAGTCTGGTGTACTCG | ATGCCCACTCGCTGATTGG |
| | (SEQ ID NO: 31) | (SEQ ID NO: 32) |
| TPSABI | GCCAATGACACCTACTGGATG | GAGCTGTACTCTGACCTTGTTG |
| | (SEQ ID NO: 33) | (SEQ ID NO: 34) |
| Taqman Assay | ABI Assay ID | |
| PPIA | Hs04194521_s1 | |
| CXCL5 | Hs00171085_m1 | |

Shown are gene ID, forward and reverse primer sequences, or assay ID for commercial assays.

### Immunohistochemistry (IHC).

Immunostaining was performed on formalin-fixed, paraffin-embedded lung tissue sections from human samples. Total mast cells numbers were identified by expression of tryptase using a tryptase antibody (mouse, anti-human antibody M7052; 1:1000; Dako; Carpinteria, Calif.) and the connective tissue mast cell sub-population (MC_{TC}) was identified by chymase expression using a chymase antibody (mouse, anti-human antibody MCA1930; 1:500; ABD Serotec; Raleigh, N.C.). The number of mast cells per field (200×) was defined in 10 random fields and was summarized as the total number of cells/field for each subject. At least eight non-BPD and 11 BPD samples were studied. Control slides were stained with either secondary antibody alone, purified IgG or pre-immune serum. For some analyses, the anatomical location of the cell (parenchyma, mucosal, perivascular, peribronchoiolar) was further defined. In this case, data were normalized for the number of specific fields containing that anatomical feature. For mouse samples, mast cell numbers were identified by chymase (Cma1;Mcp5) expression (MCA1930, 1:200, ABD Serotec), using the Mouse-on-Mouse kit (Vector Labs, Burlingame, Calif.). Generation and analysis of mice deficient in expression of both FGFR3 and FGFR4 (FGFR3/4) was performed. Whole lung tissue RNA was isolated from FGFR3/4 mutant and wild type controls at 1 month of age and subjected to qPCR analysis for CPA3, TPSAB1 and TPSB2 using gene-specific primers.

### Statistical Analysis.

Statistical analysis of microarray data was performed using standard approaches as described above. For qPCR and IHC, data were summarized independently for each subject. Group means and group variation were used to calculate significance according to the non-parametric Mann-Whitney U-test.

### Results

### Subject Demographics

From the biorepository collection, lung tissue samples from a total of 28 subjects were studied (Table 1). Eleven of these subjects died with a clinical and pathological diagnosis of BPD. Nine control subjects, matched for age at birth and death, displayed evidence of mild lung pathology (pneumonia, respiratory distress syndrome (RDS) or alveolar hemorrhage). Another 8 subjects with significant, non-BPD pulmonary pathology, primarily consisting of inflammation associated with sepsis, were used as an additional comparison group. The primary analysis was to distinguish all the BPD (n=11) subjects from all mild lung pathology controls (n=9). Sub-analyses examined gene expression patterns stratified by age, as defined by “early” (<27 weeks estimated gestational age (EGA) at birth; <35 weeks EGA at death) or “late” (>27 weeks EGA at birth; >35 weeks EGA at death) gestation. When matched for age at birth or death, only one sample was re-classified; sample 51 was defined as “late” at birth, but “early” at death.

### Pathways Affected in BPD

RNA was isolated from grossly dissected distal lung tissue and processed as described above. All RNA samples studied passed quality control assessments and were interrogated for genome-wide expression using the Affymetrix Hu133 Plus 2.0 array, essentially as previously described (Bhattacharya et al., Am. J. Respir. Cell Mol. Biol. 40:359-367 (2009); Kho et al., Am. J. Respir. Crit. Care Med. 181:54-63 (2010)). Normalized and background corrected data were extracted using RMA.

Significant diminution of vessel formation occurs in BPD lungs. Therefore, the microarray-based expression patterns of vascular molecules including cell surface proteins (PECAM, TIE2, FLT1, ENG), growth factors (VEGF, ANG1) and signaling molecules (HIF1A, HIF3A) were examined in this data set. Seven of nine genes examined (and 19 of 26 probe sets examined) demonstrated evidence for reduced expression (fold change<2) in BPD when compared to controls (Table 3). Among the 7 vascular marker genes demonstrating reduced expression, HIF3A and TIE2 were significantly reduced (p<0.05) in BPD tissue. Additionally, expression of NOS genes was examined, since NO has been implicated in BPD pathogenesis. All three NOS genes (NOS1, NOS2, NOS3) showed some evidence for decreased expression, and NOS2 was significantly reduced (p<0.02) in BPD. These data validate that the approach was able to reliably capture known molecular alterations associated with BPD pathology.

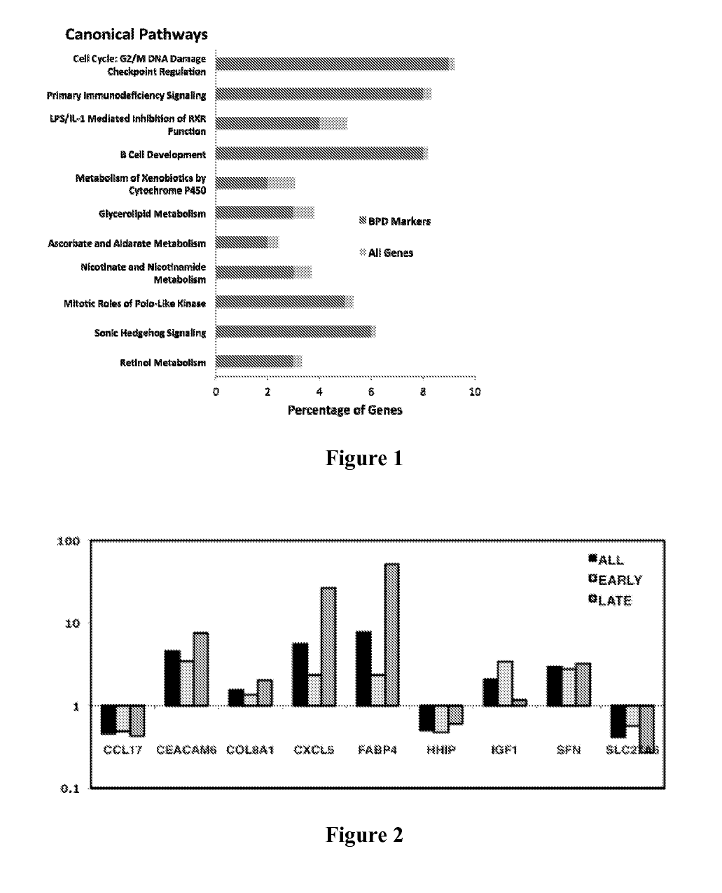

This study further focused on a set of 159 genes with significant differences in expression between BPD and controls (p<0.05; >2-fold difference) as defined by the microarray data set. A complete list of these genes is presented in Table 4. Canonical pathways over-represented in these 159 genes were tested for using Ingenuity Pathways Analysis software (FIG. 1) (Ingenuity Systems; Redwood City, Calif.). Discovered biological processes significantly associated with BPD included cell cycle regulation (DNA damage-related, Polo-like kinase) and immune cell regulation (immunodeficiency signaling, B cell development). Additionally, gene expression in BPD was associated with specific developmental and biochemical pathways, including sonic hedgehog signaling and retinol metabolism. These results demonstrated that BPD has a complex disease pathology associated with changes in cell proliferation, oxidant stress-related DNA damage-repair, inflammatory cell infiltration and ongoing developmental processes.

TABLE 3
Gene-based expression patterns of vascular molecules in BPD.

| Gene Symbol | Probeset ID | P-value | Fold Change |
|---|---|---|---|
| HIF3A | 222123_s_at | 0.051517864 | 0.499235075 |
| HIF3A | 219319_at | 0.046117395 | 0.516916465 |
| HIF3A | 222124_at | 0.081431338 | 0.54159644 |
| HIF3A | 233517_s_at | 0.093358093 | 0.542803877 |
| HIF3A | 232669_at | 0.103914538 | 0.554755041 |
| HIF3A | 1555318_at | 0.008245153 | 0.554952475 |
| HIF3A | 1556069_s_at | 0.12641404 | 0.563880546 |
| FLT1 | 204406_at | 0.09223114 | 0.771862157 |
| NOS1 | 1560974_s_at | 0.21261651 | 0.786716109 |
| NOS2 | 210037_s_at | 0.017367679 | 0.817104988 |
| TIE2 | 206702_at | 0.023569983 | 0.81958691 |
| NOS3 | 205581_s_at | 0.41164593 | 0.88274014 |
| NOS1 | 207309_at | 0.149091481 | 0.892412662 |
| NOS1 | 239132_at | 0.630650867 | 0.901441679 |
| PECAM1 | 208983_s_at | 0.307257654 | 0.905981347 |
| PECAM1 | 208982_at | 0.07038192 | 0.909589048 |
| PECAM1 | 1559921_at | 0.471753205 | 0.922816889 |
| PECAM1 | 208981_at | 0.200078132 | 0.929374932 |
| TIE2 | 217711_at | 0.232586453 | 0.929629703 |
| HIF1AN | 226648_at | 0.455298294 | 0.931352423 |
| ENG | 201808_s_at | 0.680594812 | 0.940850606 |
| VEGF | 212171_x_at | 0.665922235 | 0.941991647 |
| ENG | 201809_s_at | 0.742002371 | 0.946332992 |
| VEGF | 210512_s_at | 0.581133153 | 0.947641054 |
| NOS1 | 207310_s_at | 0.299146087 | 0.952411042 |
| VEGF | 59999_at | 0.278196965 | 0.95664521 |
| FLT1 | 218525_s_at | 0.469167701 | 0.965453072 |
| HIGD2A | 218030_at | 0.667760675 | 0.975167336 |
| HIGD2A | 232809_s_at | 0.885289397 | 0.989030128 |
| HIGD1A | 240911_at | 0.945131937 | 0.995488305 |
| ANG1 | 205608_s_at | 0.59 | 1.08 |
| HIGD1A | 210287_s_at | 0.73949446 | 1.047586412 |
| HIGD1A | 209328_x_at | 0.106842365 | 1.06248233 |
| ANG1 | 209329_x_at | 0.338908305 | 1.06289106 |
| ANG1 | 217845_x_at | 0.172427453 | 1.073999065 |
| HIF1A | 200989_at | 0.06 | 1.13 |
| VEGF | 242317_at | 0.308540229 | 1.102899948 |

TABLE 4
Complete list of 159 gene-based expression patterns

| Rank | Probeset ID | Symbol | P-value | Fold Change | Location | Type |
|---|---|---|---|---|---|---|
| 1 | 205624_at | CPA3 | 2.27E−03 | 7.250095506 | ECS | peptidase |
| 2 | 32128_at | CCL18 | 1.95E−03 | 6.727171322 | ECS | cytokine |
| 3 | 217022_s_at | IGHA1 | 1.94E−02 | 6.685335671 | ECS | other |
| 4 | 207134_x_at | TPSB2 | 5.65E−03 | 5.248845018 | ECS | peptidase |
| 5 | 205683_x_at | TPSAB1 | 4.88E−03 | 5.151526518 | ECS | peptidase |
| 6 | 203180_at | ALDH1A3 | 2.25E−03 | 4.540968409 | Cytoplasm | enzyme |
| 7 | 211430_s_at | IGHM | 8.02E−02 | 4.37414183 | PM | TMR |
| 8 | 220542_s_at | PLUNC | 3.47E−02 | 4.172754374 | ECS | other |
| 9 | 203980_at | FABP4 | 8.77E−03 | 4.158317741 | Cytoplasm | transporter |
| 10 | 232798_at | DNAJC5B | 8.24E−03 | 4.0278222 | unknown | transporter |
| 11 | 1553155_x_at | ATP6V0D2 | 8.54E−03 | 3.721798631 | unknown | transporter |
| 12 | 205242_at | CXCL13 | 3.60E−02 | 3.637633007 | ECS | cytokine |
| 13 | 205767_at | EREG | 9.83E−03 | 3.282966435 | ECS | growth factor |
| 14 | 201884_at | CEACAM5 | 1.66E−02 | 3.168939244 | PM | other |
| 15 | 205713_s_at | COMP | 9.43E−03 | 3.018851937 | ECS | other |
| 16 | 226067_at | C20ORF114 | 9.41E−02 | 2.97729051 | ECS | other |
| 17 | 212592_at | IGJ | 1.81E−02 | 2.950582914 | ECS | other |
| 18 | 206932_at | CH25H | 1.84E−02 | 2.844154821 | Cytoplasm | enzyme |
| 19 | 1557197_a_at | LGALS3 | 9.13E−03 | 2.834314793 | ECS | other |
| 20 | 33323_r_at | SFN | 2.96E−02 | 2.785622961 | Cytoplasm | other |
| 21 | 215214_at | IGL@ | 6.29E−02 | 2.783692784 | Nucleus | other |
| 22 | 214587_at | COL8A1 | 1.97E−04 | 2.718856484 | ECS | other |
| 23 | 222049_s_at | RBP4 | 5.51E−02 | 2.71508996 | ECS | transporter |
| 24 | 231702_at | TDO2 | 2.15E−02 | 2.670296607 | Cytoplasm | enzyme |
| 25 | 207067_s_at | HDC | 8.44E−03 | 2.575763259 | Cytoplasm | enzyme |
| 26 | 207430_s_at | MSMB | 1.39E−01 | 2.559744828 | ECS | other |
| 27 | 210511_s_at | INHBA | 2.22E−02 | 2.549121255 | ECS | growth factor |
| 28 | 219918_s_at | ASPM | 4.88E−03 | 2.498392251 | Nucleus | other |
| 29 | 205542_at | STEAP1 | 9.45E−03 | 2.486299338 | PM | transporter |
| 30 | 202238_s_at | NNMT | 4.02E−03 | 2.465704651 | Cytoplasm | enzyme |
| 31 | 219295_s_at | PCOLCE2 | 8.37E−02 | 2.46058269 | ECS | other |
| 32 | 215217_at | IGKC | 1.75E−01 | 2.45206972 | ECS | other |
| 33 | 219148_at | PBK | 2.11E−02 | 2.438510188 | Cytoplasm | kinase |
| 34 | 224942_at | PAPPA | 4.72E−02 | 2.421666168 | ECS | peptidase |
| 35 | 211657_at | CEACAM6 | 5.82E−04 | 2.401606855 | PM | other |
| 36 | 205758_at | CD8A | 1.16E−01 | 2.386671486 | PM | other |
| 37 | 219502_at | NEIL3 | 3.92E−03 | 2.383365149 | Nucleus | enzyme |
| 38 | 231562_at | APOC2 | 3.09E−02 | 2.352182501 | ECS | transporter |
| 39 | 206519_x_at | SIGLEC6 | 1.99E−02 | 2.339175328 | ECS | other |
| 40 | 209773_s_at | RRM2 | 5.62E−03 | 2.337554497 | Nucleus | enzyme |
| 41 | 206632_s_at | APOBEC3B | 1.06E−02 | 2.316585612 | Cytoplasm | enzyme |
| 42 | 229070_at | C6ORF105 | 3.13E−02 | 2.316585612 | unknown | other |
| 43 | 1560011_at | PSCA | 1.10E−03 | 2.314980434 | PM | other |
| 44 | 238125_at | ADAMTS16 | 2.20E−02 | 2.300583787 | ECS | other |
| 45 | 218469_at | GREM1 | 8.91E−02 | 2.292624371 | ECS | Other |
| 46 | 242809_at | IL1RL1 | 2.05E−01 | 2.289448321 | PM | TMR |
| 47 | 214844_s_at | DOK5 | 2.82E−03 | 2.286276671 | PM | Other |
| 48 | 215101_s_at | CXCL5 | 6.16E−02 | 2.286276671 | ECS | cytokine |
| 49 | 204602_at | DKK1 | 4.06E−02 | 2.283109414 | ECS | growth factor |
| 50 | 231534_at | CDK1 | 2.91E−03 | 2.281527432 | Nucleus | Kinase |
| 51 | 204637_at | CGA | 6.86E−02 | 2.279946545 | ECS | Other |
| 52 | 222958_s_at | DEPDC1 | 9.66E−03 | 2.261061134 | unknown | Other |
| 53 | 206211_at | SELE | 1.58E−01 | 2.261061134 | PM | Other |
| 54 | 205220_at | GPR109B | 3.74E−02 | 2.250116969 | PM | GPCR |
| 55 | 208168_s_at | CHIT1 | 5.80E−03 | 2.233025924 | ECS | Enzyme |
| 56 | 228703_at | P4HA3 | 1.33E−02 | 2.223758315 | unknown | Enzyme |
| 57 | 203700_s_at | DIO2 | 2.69E−03 | 2.212994706 | Cytoplasm | Enzyme |
| 58 | 221651_x_at | IGK@ | 1.47E−01 | 2.212994706 | ECS | Other |
| 59 | 230318_at | SERPINA1 | 5.78E−02 | 2.203810232 | ECS | Other |
| 60 | 204638_at | ACP5 | 7.05E−02 | 2.200757219 | Cytoplasm | phosphatase |
| 61 | 203649_s_at | PLA2G2A | 1.07E−01 | 2.199232299 | ECS | Enzyme |
| 62 | 228636_at | BHLHE22 | 1.61E−01 | 2.196185628 | Nucleus | TR |
| 63 | 204822_at | TTK | 8.40E−03 | 2.188587403 | Nucleus | kinase |
| 64 | 1554452_a_at | C7ORF68 | 2.15E−02 | 2.185555478 | unknown | other |
| 65 | 205857_at | SLC18A2 | 1.38E−02 | 2.184041091 | PM | transporter |
| 66 | 224397_s_at | TMTC1 | 2.21E−02 | 2.184041091 | unknown | other |
| 67 | 236641_at | KIF14 | 3.11E−03 | 2.177994031 | Cytoplasm | other |
| 68 | 222039_at | KIF18B | 1.93E−02 | 2.177994031 | unknown | other |
| 69 | 203963_at | CA12 | 1.66E−02 | 2.176484883 | PM | enzyme |
| 70 | 223122_s_at | SFRP2 | 1.42E−01 | 2.173469725 | PM | TMR |
| 71 | 205157_s_at | KRT17 | 1.49E−01 | 2.164449289 | Cytoplasm | other |
| 72 | 223660_at | ADORA3 | 7.77E−02 | 2.15248025 | PM | GPCR |
| 73 | 211144_x_at | TARP | 5.10E−02 | 2.146520573 | Cytoplasm | other |
| 74 | 217640_x_at | SKA1 | 8.40E−03 | 2.145033234 | unknown | other |
| 75 | 206680_at | CD5L | 3.23E−02 | 2.143546925 | PM | TMR |
| 76 | 209596_at | MXRA5 | 8.70E−02 | 2.143546925 | unknown | other |
| 77 | 203764_at | DLGAP5 | 1.16E−02 | 2.142061646 | Nucleus | phosphatase |
| 78 | 218451_at | CDCP1 | 2.31E−02 | 2.142061646 | PM | other |
| 79 | 209642_at | BUB1 | 2.74E−03 | 2.139094176 | Nucleus | kinase |
| 80 | 1552619_a_at | ANLN | 1.05E−02 | 2.137611982 | Cytoplasm | other |
| 81 | 202953_at | C1QB | 3.46E−02 | 2.137611982 | ECS | other |
| 82 | 202503_s_at | KIAA0101 | 7.27E−03 | 2.125791349 | Nucleus | other |
| 83 | 205306_x_at | KMO | 3.22E−02 | 2.125791349 | Cytoplasm | enzyme |
| 84 | 219181_at | LIPG | 1.48E−01 | 2.124318373 | ECS | enzyme |
| 85 | 205167_s_at | CDC25C | 1.18E−02 | 2.119905567 | Nucleus | phosphatase |
| 86 | 207496_at | MS4A2 | 3.05E−02 | 2.118436669 | PM | TMR |
| 87 | 220655_at | TNIP3 | 1.38E−02 | 2.112571251 | unknown | other |
| 88 | 218232_at | C1QA | 2.81E−02 | 2.099433367 | ECS | other |
| 89 | 224156_x_at | IL17RB | 1.20E−02 | 2.097978655 | PM | TMR |
| 90 | 1558972_s_at | THEMIS | 1.29E−01 | 2.096524951 | unknown | other |
| 91 | 219669_at | CD177 | 1.94E−01 | 2.096524951 | Cytoplasm | other |
| 92 | 204962_s_at | CENPA | 1.40E−02 | 2.09216988 | Nucleus | other |
| 93 | 212023_s_at | MKI67 | 1.23E−02 | 2.0907202 | Nucleus | other |
| 94 | 244427_at | KIF23 | 2.23E−03 | 2.087823855 | Cytoplasm | other |
| 95 | 219697_at | HS3ST2 | 2.48E−02 | 2.087823855 | Cytoplasm | enzyme |
| 96 | 223700_at | MND1 | 5.08E−03 | 2.084931522 | Nucleus | other |
| 97 | 209182_s_at | C10ORF10 | 5.14E−02 | 2.080600533 | Cytoplasm | other |
| 98 | 1555778_a_at | POSTN | 3.37E−02 | 2.07915887 | ECS | other |
| 99 | 209728_at | HLA-DRB4 | 4.81E−01 | 2.07915887 | PM | TMR |
| 100 | 230237_at | ADCYAP1 | 1.18E−01 | 2.077718207 | ECS | other |
| 101 | 220301_at | CCDC102B | 1.22E−02 | 2.064797071 | unknown | other |
| 102 | 220658_s_at | ARNTL2 | 2.86E−02 | 2.057653416 | Nucleus | TR |
| 103 | 219519_s_at | SIGLEC1 | 4.98E−02 | 2.054802879 | PM | other |
| 104 | 202094_at | BIRC5 | 1.76E−02 | 2.051956291 | Cytoplasm | other |
| 105 | 229538_s_at | IQGAP3 | 4.34E−02 | 2.051956291 | unknown | other |
| 106 | 210052_s_at | TPX2 | 6.15E−03 | 2.047693801 | Nucleus | other |
| 107 | 209542_x_at | IGF1 | 8.90E−03 | 2.034959384 | ECS | growth factor |
| 108 | 209891_at | SPC25 | 3.00E−02 | 2.025109615 | unknown | other |
| 109 | 209714_s_at | CDKN3 | 6.98E−03 | 2.022304162 | Nucleus | phosphatase |
| 110 | 219799_s_at | DHRS9 | 1.91E−02 | 2.020902893 | Cytoplasm | enzyme |
| 111 | 206504_at | CYP24A1 | 3.11E−02 | 2.020902893 | Cytoplasm | enzyme |
| 112 | 201291_s_at | TOP2A | 1.82E−02 | 2.018103268 | Nucleus | enzyme |
| 113 | 205266_at | LIF | 1.16E−01 | 2.018103268 | ECS | cytokine |
| 114 | 228892_at | SH3RF2 | 7.57E−03 | 2.01670491 | unknown | other |
| 115 | 213338_at | TMEM158 | 2.15E−02 | 2.01670491 | PM | other |
| 116 | 220997_s_at | DIAPH3 | 7.49E−03 | 2.011121161 | Cytoplasm | enzyme |
| 117 | 204641_at | NEK2 | 8.27E−03 | 2.008335086 | Nucleus | kinase |
| 118 | 205819_at | MARCO | 1.17E−01 | 2.001386775 | PM | TMR |
| 119 | 1569675_at | POU2AF1 | 9.41E−02 | 0.499653546 | Nucleus | TR |
| 120 | 226690_at | ADCYAP1R1 | 6.13E−02 | 0.497235084 | PM | GPCR |
| 121 | 206209_s_at | CA4 | 6.51E−04 | 0.496202187 | PM | enzyme |
| 122 | 213909_at | LRRC15 | 1.37E−01 | 0.493116352 | PM | other |
| 123 | 228806_at | RORC | 6.18E−03 | 0.486664687 | Nucleus | LDNR |
| 124 | 210081_at | AGER | 1.28E−02 | 0.484309095 | PM | TMR |
| 125 | 213992_at | COL4A6 | 7.68E−03 | 0.479964631 | ECS | other |
| 126 | 207245_at | UGT2B17 | 9.48E−02 | 0.477310507 | Cytoplasm | enzyme |
| 127 | 230469_at | RTKN2 | 9.16E−02 | 0.473356816 | PM | other |
| 128 | 221728_x_at | XIST | 5.58E−01 | 0.473028823 | Nucleus | other |
| 129 | 204913_s_at | SOX11 | 5.48E−02 | 0.47237352 | Nucleus | TR |
| 130 | 219564_at | KCNJ16 | 4.91E−02 | 0.471065637 | PM | ion channel |
| 131 | 1556037_s_at | HHIP | 3.44E−02 | 0.469761375 | PM | other |
| 132 | 204664_at | ALPP | 1.03E−02 | 0.468136124 | ECS | phosphatase |
| 133 | 210445_at | FABP6 | 9.35E−03 | 0.465870215 | Cytoplasm | transporter |
| 134 | 229125_at | KANK4 | 3.42E−04 | 0.464258426 | unknown | other |
| 135 | 219932_at | SLC27A6 | 1.02E−03 | 0.461691155 | PM | transporter |
| 136 | 222106_at | PRND | 2.65E−01 | 0.449066186 | PM | other |
| 137 | 211734_s_at | FCER1A | 1.78E−02 | 0.433168428 | PM | TMR |
| 138 | 230924_at | TTLL6 | 3.36E−04 | 0.42720487 | unknown | enzyme |
| 139 | 223862_at | GHRL | 1.26E−01 | 0.415235012 | ECS | growth factor |
| 140 | 231223_at | CSMD1 | 1.22E−02 | 0.414372452 | PM | other |
| 141 | 207861_at | CCL22 | 2.28E−02 | 0.413511684 | ECS | cytokine |
| 142 | 205969_at | AADAC | 4.54E−02 | 0.412081042 | Cytoplasm | enzyme |
| 143 | 207900_at | CCL17 | 4.50E−04 | 0.40528256 | ECS | cytokine |
| 144 | 205216_s_at | APOH | 2.54E−02 | 0.404440674 | ECS | transporter |
| 145 | 238062_at | GPIHBP1 | 7.14E−02 | 0.393653988 | unknown | other |
| 146 | 219949_at | LRRC2 | 2.16E−02 | 0.393108646 | unknown | other |
| 147 | 232193_at | GSTT1 | 1.06E−02 | 0.389852421 | Cytoplasm | enzyme |
| 148 | 219732_at | LPPR1 | 3.34E−02 | 0.385820044 | unknown | other |
| 149 | 235763_at | SLC44A5 | 9.88E−03 | 0.385552706 | unknown | other |
| 150 | 213456_at | SOSTDC1 | 1.39E−02 | 0.37579037 | ECS | other |
| 151 | 206027_at | S100A3 | 6.59E−03 | 0.371645746 | unknown | transporter |
| 152 | 206658_at | UPK3B | 9.10E−03 | 0.358737384 | PM | other |
| 153 | 211480_s_at | SLCO1A2 | 3.67E−02 | 0.333555792 | PM | transporter |
| 154 | 1554524_a_at | OLFM3 | 1.43E−02 | 0.323536414 | Cytoplasm | other |
| 155 | 203074_at | ANXA8L2 | 1.51E−02 | 0.312949115 | unknown | other |
| 156 | 215729_s_at | VGLL1 | 6.71E−03 | 0.306508715 | Nucleus | TR |
| 157 | 208399_s_at | EDN3 | 4.52E−02 | 0.282436804 | ECS | other |
| 158 | 223597_at | ITLN1 | 4.14E−02 | 0.282241101 | PM | other |
| 159 | 205048_s_at | PSPH | 8.37E−02 | 0.261340025 | unknown | phosphatase |

ECS: extracellular space; PM: plasma membrane; GPCR: G-protein coupled receptor; TMR: transmembrane receptor; LDNR: ligand-dependent nuclear receptor; TR: transcriptional regulator

BPD-Associated Gene Expression Global expression patterns for the 159 genes in all samples were analyzed in order to assess age-related changes in expression and disease-specificity. Significant variability in expression of individual genes within subject groups was apparent, as expected, given the diverse pathology associated with this disease. Even so, genes displaying consistent increases or decreases in expression in BPD, as compared to controls, were apparent. Interestingly, a majority of these genes did not display evidence for age-dependent changes in expression in controls. However, a small set of genes showed a trend for reduced expression over time in controls, but persistent expression over time in BPD tissues. The specificity of gene expression changes identified in BPD was also assessed. While most gene expression changes identified in BPD lungs were specific, a subset of genes induced in BPD tissues showed similar changes in non-BPD lung disease tissues. Another set of genes displayed similar expression patterns restricted to BPD and sepsis only.

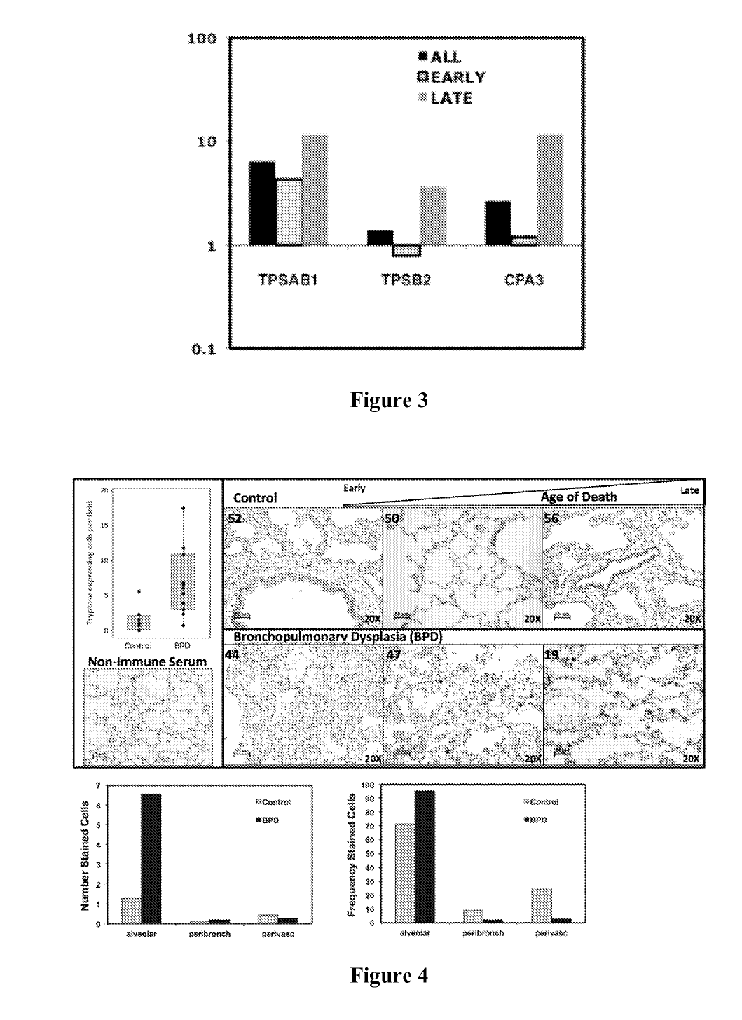

In order to further assess the reliability of the microarray data set and the methods for identifying disease gene expression biomarkers, the expression of selected genes were validated by qPCR (FIGS. 2 and 3). Genes were chosen for validation studies based upon their inclusion in the set of 159 differentially expressed genes, their magnitude of change in expression, and interest in their potential biological function. In addition to comparing all BPD and all controls, the analysis was stratified by estimated gestational age (EGA) to compare disease-related patterns in subjects earlier in age (<27 weeks EGA at birth; <35 weeks EGA at death) or later in age (>27 weeks EGA at birth; >35 weeks EGA at death)

A significant difference (P<0.05) in expression between BPD and controls was confirmed for 8 out of a total of 13 genes tested including CCL17, CEACAM6, COL8A1, CXCL5, FABP4, HHIP, IGF1, SFN, SLC27A6. Other genes showed some evidence for differential expression by qPCR, consistent with the microarray results. CXCL5 showed a 5-fold increase by qPCR and was significantly increased in “late” stage BPD samples. CPA3 showed a ˜3-fold increase and was significantly increased in “late” stage BPD samples (GAB, trend GAD). CCL17 was decrease 2-fold and demonstrated a trend for significance in all samples (P=0.06). TPSB2 was increased 3.5-fold in “late” stage BPD samples. HHIP showed a 2-fold reduction in all samples.

TABLE 5
Genes significantly affected in BPD lung tissue.

| Genes | Probeset | P Value | Fold Change |
|---|---|---|---|
| CPA3 | 205624_at | 2.27E−03 | 7.25 |
| CCL18 | 32128_at | 1.95E−03 | 6.73 |
| IGHA1 | 217022_s_at | 1.94E−02 | 6.69 |
| TPSB2 | 207134_x_at | 5.65E−03 | 5.25 |
| TPSAB1 | 205683_x_at | 4.88E−03 | 5.15 |
| ALDH1A3 | 203180_at | 2.25E−03 | 4.54 |
| IGHM | 211430_s_at | 8.02E−02 | 4.37 |
| PLUNC | 220542_s_at | 3.47E−02 | 4.17 |
| FABP4 | 203980_at | 8.77E−03 | 4.16 |
| DNAJC5B | 232798_at | 8.24E−03 | 4.03 |
| ATP6V0D2 | 1553155_x_at | 8.54E−03 | 3.72 |
| CXCL13 | 205242_at | 3.60E−02 | 3.64 |
| EREG | 205767_at | 9.83E−03 | 3.28 |
| CEACAM5 | 201884_at | 1.66E−02 | 3.17 |
| COMP | 205713_s_at | 9.43E−03 | 3.02 |
| C20ORF114 | 226067_at | 9.41E−02 | 2.98 |
| IGJ | 212592_at | 1.81E−02 | 2.95 |
| CH25H | 206932_at | 1.84E−02 | 2.84 |
| LGALS3 | 1557197_a_at | 9.13E−03 | 2.83 |
| SFN | 33323_r_at | 2.96E−02 | 2.79 |
| IGL@ | 215214_at | 6.29E−02 | 2.78 |
| COL8A1 | 214587_at | 1.97E−04 | 2.72 |
| RBP4 | 222049_s_at | 5.51E−02 | 2.72 |
| TDO2 | 231702_at | 2.15E−02 | 2.67 |
| HDC | 207067_s_at | 8.44E−03 | 2.58 |
| MSMB | 207430_s_at | 1.39E−01 | 2.56 |
| INHBA | 210511_s_at | 2.22E−02 | 2.55 |
| ASPM | 219918_s_at | 4.88E−03 | 2.50 |
| STEAP1 | 205542_at | 9.45E−03 | 2.49 |
| NNMT | 202238_s_at | 4.02E−03 | 2.47 |
| PCOLCE2 | 219295_s_at | 8.37E−02 | 2.46 |
| IGKC | 215217_at | 1.75E−01 | 2.45 |
| PBK | 219148_at | 2.11E−02 | 2.44 |
| PAPPA | 224942_at | 4.72E−02 | 2.42 |
| CEACAM6 | 211657_at | 5.82E−04 | 2.40 |
| CD8A | 205758_at | 1.16E−01 | 2.39 |
| NEIL3 | 219502_at | 3.92E−03 | 2.38 |
| APOC2 | 231562_at | 3.09E−02 | 2.35 |
| SIGLEC6 | 206519_x_at | 1.99E−02 | 2.34 |
| RRM2 | 209773_s_at | 5.62E−03 | 2.34 |
| APOBEC3B | 206632_s_at | 1.06E−02 | 2.32 |
| C6ORF105 | 229070_at | 3.13E−02 | 2.32 |
| PSCA | 1560011_at | 1.10E−03 | 2.31 |
| ADAMTS16 | 238125_at | 2.20E−02 | 2.30 |
| GREM1 | 218469_at | 8.91E−02 | 2.29 |
| IL1RL1 | 242809_at | 2.05E−01 | 2.29 |
| DOK5 | 214844_s_at | 2.82E−03 | 2.29 |
| CXCL5 | 215101_s_at | 6.16E−02 | 2.29 |
| DKK1 | 204602_at | 4.06E−02 | 2.28 |
| CDK1 | 231534_at | 2.91E−03 | 2.28 |
| CGA | 204637_at | 6.86E−02 | 2.28 |
| DEPDC1 | 222958_s_at | 9.66E−03 | 2.26 |
| SELE | 206211_at | 1.58E−01 | 2.26 |
| GPR109B | 205220_at | 3.74E−02 | 2.25 |
| CHIT1 | 208168_s_at | 5.80E−03 | 2.23 |
| P4HA3 | 228703_at | 1.33E−02 | 2.22 |
| DIO2 | 203700_s_at | 2.69E−03 | 2.21 |
| IGK@ | 221651_x_at | 1.47E−01 | 2.21 |
| SERPINA1 | 230318_at | 5.78E−02 | 2.20 |
| ACP5 | 204638_at | 7.05E−02 | 2.20 |
| PLA2G2A | 203649_s_at | 1.07E−01 | 2.20 |
| BHLHE22 | 228636_at | 1.61E−01 | 2.20 |
| TTK | 204822_at | 8.40E−03 | 2.19 |
| C7ORF68 | 1554452_a_at | 2.15E−02 | 2.19 |
| SLC18A2 | 205857_at | 1.38E−02 | 2.18 |
| TMTC1 | 224397_s_at | 2.21E−02 | 2.18 |
| KIF14 | 236641_at | 3.11E−03 | 2.18 |
| KIF18B | 222039_at | 1.93E−02 | 2.18 |
| CA12 | 203963_at | 1.66E−02 | 2.18 |
| SFRP2 | 223122_s_at | 1.42E−01 | 2.17 |
| KRT17 | 205157_s_at | 1.49E−01 | 2.16 |
| ADORA3 | 223660_at | 7.77E−02 | 2.15 |
| TARP | 211144_x_at | 5.10E−02 | 2.15 |
| SKA1 | 217640_x_at | 8.40E−03 | 2.15 |
| CD5L | 206680_at | 3.23E−02 | 2.14 |
| MXRA5 | 209596_at | 8.70E−02 | 2.14 |
| DLGAP5 | 203764_at | 1.16E−02 | 2.14 |
| CDCP1 | 218451_at | 2.31E−02 | 2.14 |
| BUB1 | 209642_at | 2.74E−03 | 2.14 |
| ANLN | 1552619_a_at | 1.05E−02 | 2.14 |
| C1QB | 202953_at | 3.46E−02 | 2.14 |
| KIAA0101 | 202503_s_at | 7.27E−03 | 2.13 |
| KMO | 205306_x_at | 3.22E−02 | 2.13 |
| LIPG | 219181_at | 1.48E−01 | 2.12 |
| CDC25C | 205167_s_at | 1.18E−02 | 2.12 |
| MS4A2 | 207496_at | 3.05E−02 | 2.12 |
| TNIP3 | 220655_at | 1.38E−02 | 2.11 |
| C1QA | 218232_at | 2.81E−02 | 2.10 |
| IL17RB | 224156_x_at | 1.20E−02 | 2.10 |
| THEMIS | 1558972_s_at | 1.29E−01 | 2.10 |
| CD177 | 219669_at | 1.94E−01 | 2.10 |
| CENPA | 204962_s_at | 1.40E−02 | 2.09 |
| MKI67 | 212023_s_at | 1.23E−02 | 2.09 |
| KIF23 | 244427_at | 2.23E−03 | 2.09 |
| HS3ST2 | 219697_at | 2.48E−02 | 2.09 |
| MND1 | 223700_at | 5.08E−03 | 2.08 |
| C10ORF10 | 209182_s_at | 5.14E−02 | 2.08 |
| POSTN | 1555778_a_at | 3.37E−02 | 2.08 |
| HLA-DRB4 | 209728_at | 4.81E−01 | 2.08 |
| ADCYAP1 | 230237_at | 1.18E−01 | 2.08 |
| CCDC102B | 220301_at | 1.22E−02 | 2.06 |
| ARNTL2 | 220658_s_at | 2.86E−02 | 2.06 |
| SIGLEC1 | 219519_s_at | 4.98E−02 | 2.05 |
| BIRC5 | 202094_at | 1.76E−02 | 2.05 |
| IQGAP3 | 229538_s_at | 4.34E−02 | 2.05 |
| TPX2 | 210052_s_at | 6.15E−03 | 2.05 |
| IGF1 | 209542_x_at | 8.90E−03 | 2.03 |
| SPC25 | 209891_at | 3.00E−02 | 2.03 |
| CDKN3 | 209714_s_at | 6.98E−03 | 2.02 |
| DHRS9 | 219799_s_at | 1.91E−02 | 2.02 |
| CYP24A1 | 206504_at | 3.11E−02 | 2.02 |
| TOP2A | 201291_s_at | 1.82E−02 | 2.02 |
| LIF | 205266_at | 1.16E−01 | 2.02 |
| SH3RF2 | 228892_at | 7.57E−03 | 2.02 |
| TMEM158 | 213338_at | 2.15E−02 | 2.02 |
| DIAPH3 | 220997_s_at | 7.49E−03 | 2.01 |
| NEK2 | 204641_at | 8.27E−03 | 2.01 |
| MARCO | 205819_at | 1.17E−01 | 2.00 |
| POU2AF1 | 1569675_at | 9.41E−02 | 0.50 |
| ADCYAP1R1 | 226690_at | 6.13E−02 | 0.50 |
| CA4 | 206209_s_at | 6.51E−04 | 0.50 |
| LRRC15 | 213909_at | 1.37E−01 | 0.49 |
| RORC | 228806_at | 6.18E−03 | 0.49 |
| AGER | 210081_at | 1.28E−02 | 0.48 |
| COL4A6 | 213992_at | 7.68E−03 | 0.48 |
| UGT2B17 | 207245_at | 9.48E−02 | 0.48 |
| RTKN2 | 230469_at | 9.16E−02 | 0.47 |
| XIST | 221728_x_at | 5.58E−01 | 0.47 |
| SOX11 | 204913_s_at | 5.48E−02 | 0.47 |
| KCNJ16 | 219564_at | 4.91E−02 | 0.47 |
| HHIP | 1556037_s_at | 3.44E−02 | 0.47 |
| ALPP | 204664_at | 1.03E−02 | 0.47 |
| FABP6 | 210445_at | 9.35E−03 | 0.47 |
| KANK4 | 229125_at | 3.42E−04 | 0.46 |
| SLC27A6 | 219932_at | 1.02E−03 | 0.46 |
| PRND | 222106_at | 2.65E−01 | 0.45 |
| FCER1A | 211734_s_at | 1.78E−02 | 0.43 |
| TTLL6 | 230924_at | 3.36E−04 | 0.43 |
| GHRL | 223862_at | 1.26E−01 | 0.42 |
| CSMD1 | 231223_at | 1.22E−02 | 0.41 |
| CCL22 | 207861_at | 2.28E−02 | 0.41 |
| AADAC | 205969_at | 4.54E−02 | 0.41 |
| CCL17 | 207900_at | 4.50E−04 | 0.41 |
| APOH | 205216_s_at | 2.54E−02 | 0.40 |
| GPIHBP1 | 238062_at | 7.14E−02 | 0.39 |
| LRRC2 | 219949_at | 2.16E−02 | 0.39 |
| GSTT1 | 232193_at | 1.06E−02 | 0.39 |
| LPPR1 | 219732_at | 3.34E−02 | 0.39 |
| SLC44A5 | 235763_at | 9.88E−03 | 0.39 |
| SOSTDC1 | 213456_at | 1.39E−02 | 0.38 |
| S100A3 | 206027_at | 6.59E−03 | 0.37 |
| UPK3B | 206658_at | 9.10E−03 | 0.36 |
| SLCO1A2 | 211480_s_at | 3.67E−02 | 0.33 |
| OLFM3 | 1554524_a_at | 1.43E−02 | 0.32 |
| ANXA8L2 | 203074_at | 1.51E−02 | 0.31 |
| VGLL1 | 215729_s_at | 6.71E−03 | 0.31 |
| EDN3 | 208399_s_at | 4.52E−02 | 0.28 |
| ITLN1 | 223597_at | 4.14E−02 | 0.28 |
| PSPH | 205048_s_at | 8.37E−02 | 0.26 |

Shown are gene symbol, probe set ID, fold-change (BPD/control) and p-value as defined by student's t-test, for 159 genes.

The expression of KITLG, BLP and IL4 was also tested, as these have previously been reported to be altered in BPD. The results are shown in Table 5. No difference was observed in the expression of these genes in the microarray data set. IL4 expression was virtually undetectable in lung tissue samples by qPCR (CT>35). KITLG and BLP showed appreciable expression levels but did not show any significant changes in their expression by qPCR.

TABLE 5
qPCR validation of IL4, GRP and KITLG
expression in human lung tissue

| | Control | BPD | |
|---|---|---|---|
| IL4 | ND | ND | not detectable |
| GRP | 1.26 (±0.96) | 1.70 (±1.28) | p >> 0.05 |
| KITLG | 1.21 (±0.67) | 1.06 (±0.55) | p >> 0.05 |

Shown are mean fold-change (± standard deviation) in each group, non-BPD control (CTL) or BPD, relative to the control population. IL4 expression was not detectable in lung tissue from either group. GRP and KITLG expression were detectable in all lung tissues, but no differences in BPD subjects were observed.

### Identification of Mast Cell Gene Signature

Among this list of 159 BPD-associated genes, those genes with the greatest magnitude of change were examined (Table 6). Three of the top 5 genes, and 9 of the top 26 probe sets, were mast cell specific markers, suggesting an increase in mast cells in BPD tissues. Interestingly, the most highly induced gene encodes CPA3, a marker specific for connective tissue-type mast cells (MC_{TC}), a sub-population of mast cells that are not frequently found within the lung.

qPCR for mast cell specific marker expression, for both mucosal-type mast cells (MC_{T}) (TPSAB1, TPSB2) and MC_{TC} (CPA3) populations, was consistent with increases in these cells, particularly in “late” stage BPD samples (FIG. 3). CPA3 showed a ˜3-fold increase and was significantly increased in “late” stage BPD samples. TPSB2 was increased 3.5-fold in “late” stage BPD samples.

In order to confirm the gene expression data and more thoroughly to assess the predicted increase in mast cells in BPD lungs, immunohistochemistry was performed for mast cell markers (FIG. 4) Immunostaining for tryptase, a marker for all mast cell sub-types, confirmed a significant increase in total mast cell number in our BPD subjects (6.8 vs. 1.5 cells/field, p=0.005).

TABLE 6
Ranked list (based upon fold-change between BPD and control) of 25
most affected genes among 159 significantly dysregulated in BPD.

| Gene | | | | Fold |
|---|---|---|---|---|
| Symbol | Gene Title | Probeset ID | P-value | Change |
| CPA3 | carboxypeptidase A3 (mast cell) | 205624_at | 0.00 | 7.14 |
| CCL18 | chemokine (C-C motif) ligand 18 (pulmonary and | 32128_at | 0.00 | 6.74 |
| | activation-regulated) | | | |
| IGHA1 | immunoglobulin heavy constant alpha 1 | 217022_s_at | 0.02 | 6.90 |
| CCL18 | chemokine (C-C motif) ligand 18 (pulmonary and | 209924_at | 0.00 | 6.27 |
| | activation-regulated) | | | |
| TPSAB1 | tryptase alpha/beta 1 | 216474_x_at | 0.01 | 6.16 |
| TPSAB1 | tryptase alpha/beta 1 | 207134_x_at | 0.01 | 5.15 |
| TPSAB1 | tryptase alpha/beta 1 | 205683_x_at | 0.00 | 5.03 |
| ALDH1A3 | aldehyde dehydrogenase 1 family, member A3 | 203180_at | 0.00 | 4.60 |
| TPSAB1 | tryptase alpha/beta 1 | 217023_x_at | 0.01 | 4.38 |
| TPSAB1 | tryptase alpha/beta 1 | 210084_x_at | 0.01 | 4.34 |
| FABP4 | Fatty acid binding protein 4, adipocyte | 235978_at | 0.01 | 3.87 |
| PLUNC | palate, lung and nasal epithelium carcinoma | 220542_s_at | 0.03 | 4.28 |
| | associated | | | |
| FABP4 | fatty acid binding protein 4, adipocyte | 203980_at | 0.01 | 4.28 |
| DNAJC5B | DnaJ (Hsp40) homolog, subfamily C, member 5 | 232798_at | 0.01 | 4.14 |
| | beta | | | |
| TPSB2 | tryptase alpha/beta 1 /// tryptase beta 2 | 207741_x_at | 0.01 | 4.10 |
| ATP6V0D2 | ATPase, H+ transporting, lysosomal 38 kDa, V0 | 1553153_at | 0.01 | 3.82 |
| | subunit d2 | | | |
| ATP6V0D2 | ATPase, H+ transporting, lysosomal 38 kDa, V0 | 1553155_x_at | 0.01 | 3.78 |
| | subunit d2 | | | |
| CXCL13 | chemokine (C-X-C motif) ligand 13 (B-cell | 205242_at | 0.04 | 3.66 |
| | chemoattractant) | | | |
| C1orf186 | chromosome 1 open reading frame 186 | 230381_at | 0.00 | 3.82 |
| MEG8 | Maternally expressed (in Callipyge) 8 | 240083_at | 0.00 | 3.45 |
| TPSAB1 | tryptase alpha/beta 1 | 215382_x_at | 0.01 | 3.64 |
| VSIG1 | V-set and immunoglobulin domain containing 1 | 243764_at | 0.02 | 3.45 |
| DNAJC5B | DnaJ (Hsp40) homolog, subfamily C, member 5 | 1554258_a_at | 0.01 | 3.36 |
| | beta | | | |
| EREG | epiregulin | 205767_at | 0.01 | 3.30 |
| TPSAB1 | tryptase alpha/beta 1 | 216485_s_at | 0.01 | 3.29 |

Increased Connective Tissue Mast Cells are associated with BPD

As mentioned above, mast cell sub-types in the lung have been appreciated, each with different anatomical distributions and characteristic secretory products. Therefore, the distribution of tryptase expressing cells in 1) the parenchymal region of the lung, 2) adjacent to the airways and 3) adjacent to the large/intermediate vasculature was assessed. Region-specific differences between BPD and control tissues were tested. A significant increase in tryptase staining cells in BPD in the parenchymal region (6.5 vs. 1.3 cells/field, p=0.004) only, with no differences in the airway or vascular regions was observed. Interestingly, at this early developmental stage, a majority of tryptase-staining cells was observed in the parenchymal region, and not the airway, for both groups. However, these data are confounded by the proportion of tissue occupied by each region, even though the number of fields in each subject were normalized with each morphological component (e.g., parenchyma, airway, vessel).

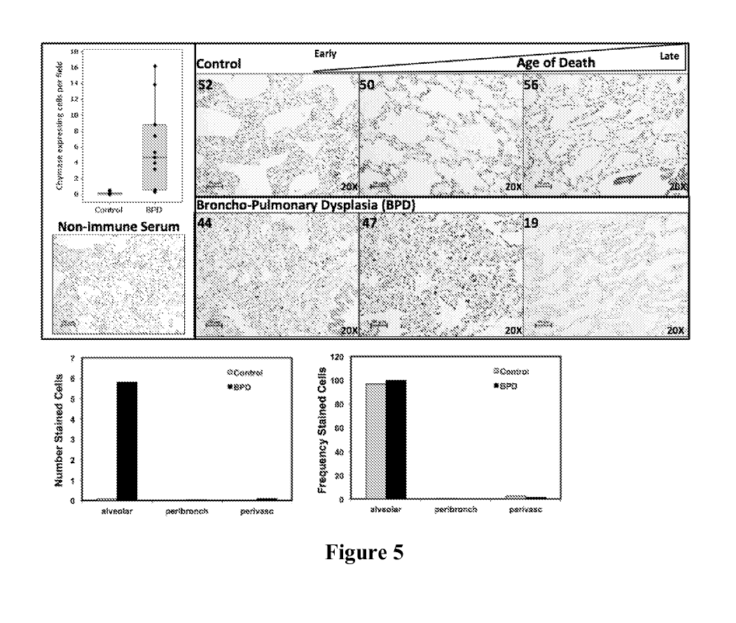

Gene expression profiling identified both mucosal mast cell (tryptase) and MC_{TC}-specific (CPA3) markers increased in BPD tissue. Furthermore, excessive tryptase-expressing cell accumulation was observed predominantly in the parenchymal region of the lungs in BPD, a location where connective tissue mast cells are typically found. Therefore, immunostaining for chymase, a MC_{TC}-specific marker, was performed (FIG. 5). A >50-fold increase in chymase-expressing cells in BPD was observed (0.1 vs. 5.9 cells/field, p=0.0003). As expected, the chymase expressing MC_{TC} mast cells were highly restricted to the parenchymal region (97% of all positive cells in BPD, 100% of all positive cells in non-BPD).

Using an independent cohort of subjects obtained from Cincinnati Children's Hospital Medical Center, we validated that increases in MCTC accumulation were not restricted to the original study cohort. Histopathological specimens from four subjects with a diagnosis of BPD were analyzed, each of which showed a frequency of MCTC (1.3, 1.9, 2.1, 2.4 chymase-expressing cells/field) substantially above controls (0.0-0.5 chymase-expressing cells/field). These data demonstrate that the specific accumulation of MCTC represents a general, and previously unappreciated, aspect of BPD pathology.

### Increased Expression of MCTC Markers in a Mouse Model of BPD-Like Pathology.

Lung development abnormalities are often present in mice lacking expression of both Fibroblast Growth Factor Receptor (FGF)-3 and -4. The FGFR3/4 mutant mouse phenocopies certain aspects of human BPD pathology including chronic lung disease characterized by structural changes in distal lung architecture consistent with developmental arrest, dysplastic elastin fiber formation and alveolar enlargement. Interestingly, under-appreciated proximal airway phenotypes in this model were detected that are reminiscent of pathology observed in individuals with BPD (squamous changes to conducting airway epithelium, bronchiolar exudates). Critically, mild monocytic infiltrates were detected in both the proximal and distal airways. Given the observations demonstrating robust accumulation of MCTC in human BPD lung samples and the presence of BPD-like pathologies in the FGFR3/4 mutant mouse lungs, the samples were tested for the presence of mast cells. Similar to the observations in human lung tissue from subjects with BPD, significant increases were noted in the expression of both general mast cell (tryptase) markers TPSAB1 (4-fold, p<0.05) and TPSB2 (3.5-fold, p=0.05), and the connective tissue-type mast cell marker CPA3 (4-fold increase, p<0.05), at 1 month of age in the FGFR3/4 mice (FIG. 3) Immunostaining for chymase supported these data, showing immunoreactive cells in airway walls and in distal airspaces of mutant mice. Dramatically fewer total inflammatory cells and chymase staining cells were observed in control mice.

### Example 2

### Identification of Chymase in Tracheal aspirates of Prematurely Born Infants

### Materials and Methods

Sample Acquisition: Samples were obtained following parents' informed, written, permission. Tracheal aspirate sample was collected by sterile technique during routine suctioning by the bedside nurse. Mechanical ventilation lungs continued through the suctioning procedure as per NICU protocol. Normal saline (0.5 ml) was instilled into the endotracheal tube through the side port of an in-line Ballard suction device. Two to five breaths later, the effluent was suctioned from the endotracheal tube into a Leukens trap. Suction catheter tip did not extend beyond tip of endotracheal tube. Infant stability was assured and steps 3-5 were repeated. The suction catheter was rinsed into the Leukens trap with an additional 1.0 mL saline. The resulting material in the Leukens trap constituted a sample. Optionally, suctioning was done without saline instillation. In this case, the suction catheter was rinsed with 2 mL saline after the suctioning procedure. The Leukens trap was labeled and capped, removing the suction tubing. The trap was placed in a refrigerator (40 C) until the sample was processed.

Sample Processing: Samples were processed within 24 hrs of collection. Contents of Leuken's trap were decanted into a 15 ml conical tube. Sample was centrifuged at 3000 rpm for 5 minutes. The supernatant was collected by pipetting into cryovials. Each cryovial aliquot was between 0.5-1 mL supernatant. The cryovials were labeled with printed subject specific study ID number and date. The supernatant aliquots were maintained in -800C freezer until assayed. Pellets from the centrifugation were processed onto slides by cytospin technique for future histology or immunohistochemistry.

Chymase Assay: The gene product was a chymotryptic serine proteinase that belongs to the peptidase family S1. It is expressed in mast cells and thought to function in the degradation of the extracellular matrix, the regulation of submucosal gland secretion, and the generation of vasoactive peptides. In the heart and blood vessels, this protein, rather than angiotensin converting enzyme, is largely responsible for converting angiotensin Ito the vasoactive peptide angiotensin II. Angiotensin II has been implicated in blood pressure control and in the pathogenesis of hypertension, cardiac hypertrophy, and heart failure. Thus, this gene product is a target for cardiovascular disease therapies. This gene maps to 14q11.2 in a cluster of genes encoding other proteases.

An aliquot of the tracheal aspirate sample supernatant was removed from the freezer, thawed to room temperature and further aliquoted and assayed utilizing the commercial Human Mast Cell Chymase ELISA kit purchased from MyBioSource.com, catalog # MBS704628 (MyBioSource. com; San Diego, Calif.). The assay was performed as directed by the supplier. In order to bring samples into the linear range of the assay, they were diluted 10-50 fold with Sample Diluent.

### Results

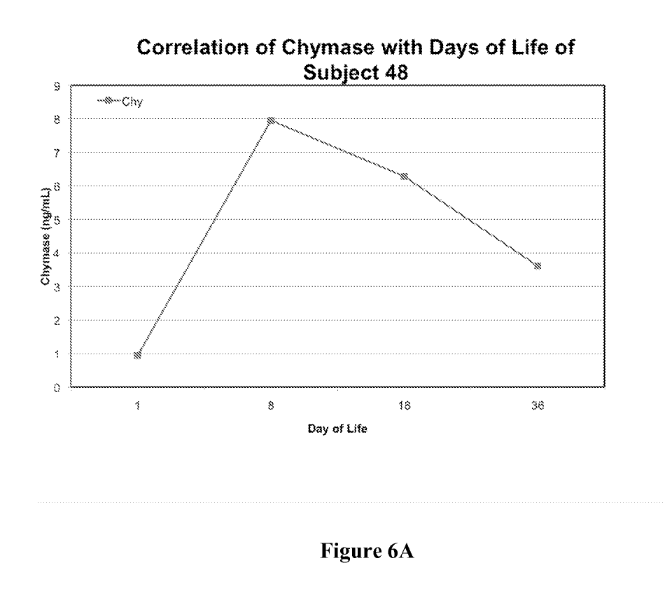

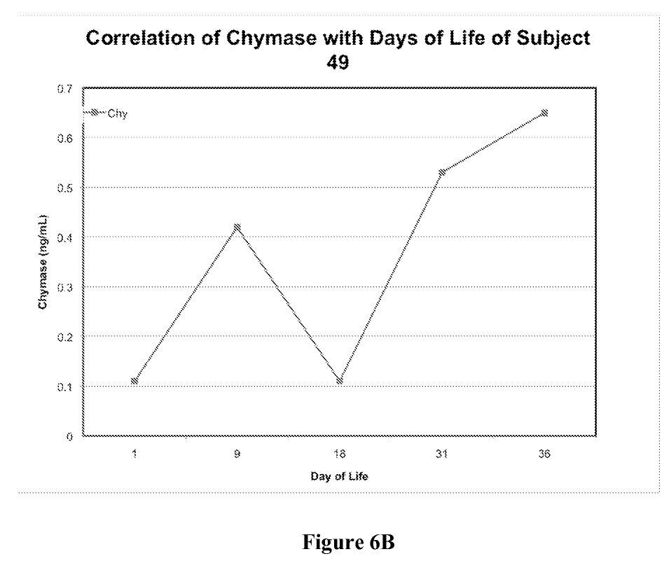

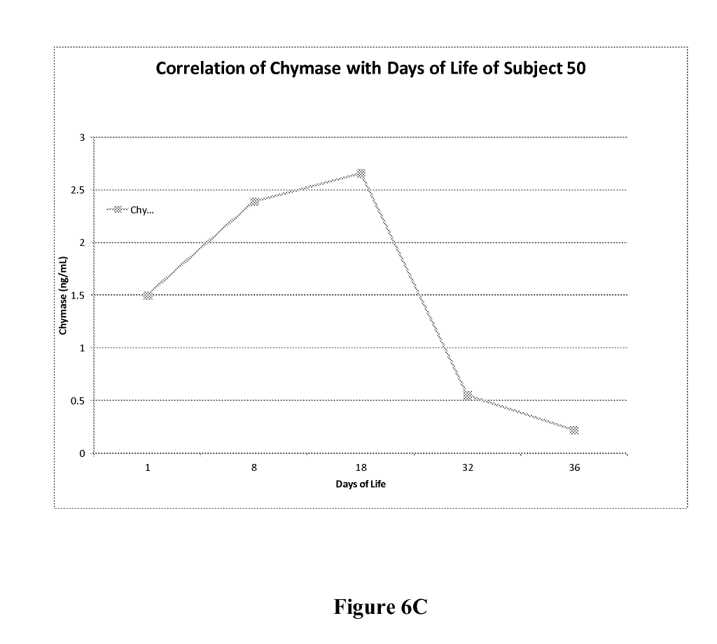

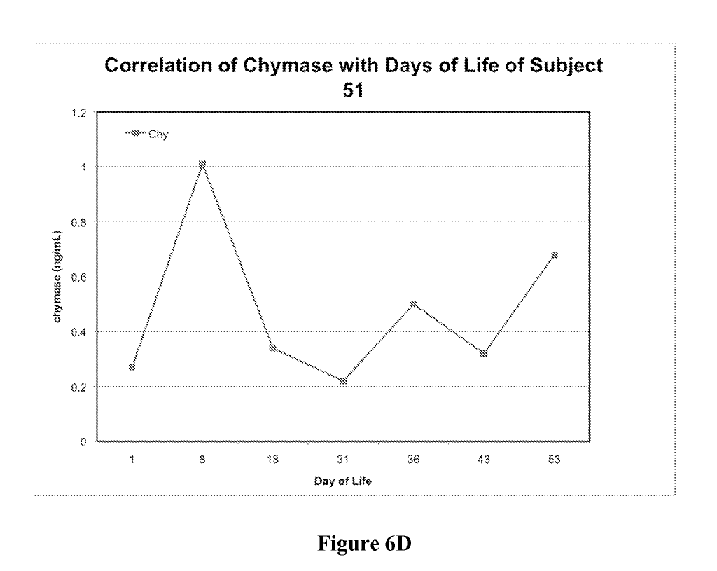

Serial tracheal aspirates were collected from a set of quadruplets born at less than 29 weeks. Chymase was detected by ELISA in each sample and is presented relative to the day after birth on which the sample was collected. Unique patterns of tracheal aspirate chymase content were detected in each infant (FIGS. 6A-D).

1. A method of identifying a subject with or at risk for developing bronchopulmoary dysplasia (BPD), the method comprising detecting chymase-expressing connective tissue mast cells (CTMCs) in a biological sample from the subject, wherein the presence of chymase-expressing CTMCs as compared to a control indicates the subject has BPD.

2. The method of claim 1, wherein the biological sample is selected from the group consisting of blood, airway aspirate, airway scrapping, brochoalveolar lavage (BAL), urine, and lung tissue.

3. The method of claim 1, wherein detecting a level of chymase-expressing CTMCs comprises detecting expression of one or more biomarkers consisting of chymase or carboxypeptidase A3.

4. The method of claim 3 further comprising detecting expression of one or more biomarkers selected from the group consisting of tryptase beta 2 (TPSB2), tryptase alpha/beta 1 (TPSAB1), cathepsin G (CTSG), and prostaglandin D2 (PGD2).

5. The method of claim 3, wherein the level of expression is detected by the level of RNA or polypeptide.

6. The method of claim 5, wherein the level of RNA is determined using an assay selected from the group consisting of a microarray analysis, a gene chip, a Northern blot, an in situ hybridization assay, a RT-PCR assay, a one step PCR assay, and a quantitative real time (qRT)-PCR assay.

7. The method of claim 5, wherein the level of polypeptide is determined using an assay selected from the group consisting of a Western blot, an enzyme-linked immunosorbent assay (ELISA), an enzyme immunoassay (EIA), a radioimmunoassay (RIA), an immunohistochemistry (IHC) assay, and a protein array.

8. A method for determining the effectiveness of a treatment regime for bronchopulmonary dysplasia (BPD) in a subject, the method comprising:
(a) identifying a first level of chymase-expressing connective tissue mast cells (CTMCs) in a first biological sample from the subject before administration of a treatment regime to the subject;
(b) identifying a second level of chymase-expressing CTMCs in a second biological sample from the subject after administration of a treatment regime to the subject;
(c) comparing the first and second level of chymase-expressing CTMCs; and
(d) adjusting the treatment regime if the second level of chymase-expressing connective tissue mast cells is the same or higher than the first level.

9. An assay system comprising a microarray or a gene chip with at least two selective binding agent, wherein each selective binding agent is specific for a biomarker selected from the group consisting of chymase, carboxypeptidase A3 (CPA3), tryptase beta 2 (TPSB2), tryptase alpha/beta 1 (TPSAB1), cathepsin G (CTSG), and prostaglandin D2 (PGD2).

10. A method for preventing or treating bronchopulmonary dysplasia (BPD) in a subject, the method comprising:
(a) identifying chymase-expressing connective tissue mast cells (CTMCs) in a biological sample from the subject; and
(b) administering to the subject an agent that blocks an activity of, blocks an increase in a level of, or reduces a level of chymase-expressing CTMCs in the subject.

11. The method of claim 10, wherein the chymase-expressing CTMCs express an increased level of carboxypeptidase A3 (CPA3) mRNA as compared to a control mast cell.

12. The method of claim 10, wherein the agent blocks expression or activity of CPA3 or chymase.

13. The method of claim 12, wherein the agent is an antibody to CPA3 or chymase.

14. The method of claim 12, wherein the agent is selected from the group consisting of a small molecule, a polypeptide, a nucleic acid, or a peptidomimetic.

15. The method of claim 14, wherein the nucleic acid is selected from the group consisting of a small interfering RNA (siRNA), a microRNA (miRNA), or an antisense nucleic acid.

16. The method of claim 10, wherein the chymase-expressing CTMCs express an increased level of one or more biomarkers selected from the group consisting of carboxypeptidase A3 (CPA3), tryptase beta 2 (TPSB2), tryptase alpha/beta 1 (TPSAB1), cathepsin G (CTSG), and prostaglandin D2 (PGD2).


SEQUENCE LISTING (34 sequences)

SEQ ID NO: 1 - DNA, 21 bp, Artificial Sequence
  Synthetic construct
cccaccgtgttcttcgacatt

SEQ ID NO: 2 - DNA, 22 bp, Artificial Sequence
  Synthetic construct
ggacccgtatgctttaggatga

SEQ ID NO: 3 - DNA, 21 bp, Artificial Sequence
  Synthetic construct
atgctcttcagttcgtgtgtg

SEQ ID NO: 4 - DNA, 21 bp, Artificial Sequence
  Synthetic construct
gggctgatacttctgggtctt

SEQ ID NO: 5 - DNA, 20 bp, Artificial Sequence
  Synthetic construct
cttctctgcagcacatccac

SEQ ID NO: 6 - DNA, 20 bp, Artificial Sequence
  Synthetic construct
ctgccctgcacagttacaaa

SEQ ID NO: 7 - DNA, 21 bp, Artificial Sequence
  Synthetic construct
cccagactcgtcttaccttgc

SEQ ID NO: 8 - DNA, 23 bp, Artificial Sequence
  Synthetic construct
ttggcgataaagagaacttgtgt

SEQ ID NO: 9 - DNA, 21 bp, Artificial Sequence
  Synthetic construct
tccccctcaaaggccaattac

SEQ ID NO: 10 - DNA, 22 bp, Artificial Sequence
  Synthetic construct
tggaacgtcccattgataaacc

SEQ ID NO: 11 - DNA, 20 bp, Artificial Sequence
  Synthetic construct
actccgtatgccatttccaa

SEQ ID NO: 12 - DNA, 21 bp, Artificial Sequence
  Synthetic construct
ttggagaactttgtcgctacc

SEQ ID NO: 13 - DNA, 22 bp, Artificial Sequence
  Synthetic construct
agcaccataaccttagatgggg

SEQ ID NO: 14 - DNA, 20 bp, Artificial Sequence
  Synthetic construct
cgtggaagtgacgcctttca

SEQ ID NO: 15 - DNA, 19 bp, Artificial Sequence
  Synthetic construct
aggccgaacgctatgagga

SEQ ID NO: 16 - DNA, 19 bp, Artificial Sequence
  Synthetic construct
ggtttcgctcttcgcagga

SEQ ID NO: 17 - DNA, 20 bp, Artificial Sequence
  Synthetic construct
gctgccacctcaaattcctc

SEQ ID NO: 18 - DNA, 19 bp, Artificial Sequence
  Synthetic construct
caggggctggtattgtgga

SEQ ID NO: 19 - DNA, 19 bp, Artificial Sequence
  Synthetic construct
ctttggccctgacggcttt

SEQ ID NO: 20 - DNA, 22 bp, Artificial Sequence
  Synthetic construct
gtagcactgagcctgtgaaatc

SEQ ID NO: 21 - DNA, 20 bp, Artificial Sequence
  Synthetic construct
tgccaccctggtttttacgg

SEQ ID NO: 22 - DNA, 21 bp, Artificial Sequence
  Synthetic construct
ttggaagcgatcacacatctc

SEQ ID NO: 23 - DNA, 22 bp, Artificial Sequence
  Synthetic construct
gggtttgattgctaccactctt

SEQ ID NO: 24 - DNA, 23 bp, Artificial Sequence
  Synthetic construct
gccaagtcctttatgatgtctgc

SEQ ID NO: 25 - DNA, 19 bp, Artificial Sequence
  Synthetic construct
ccgcgaccgatactggatg

SEQ ID NO: 26 - DNA, 20 bp, Artificial Sequence
  Synthetic construct
gatctgggcggtgtagaact

SEQ ID NO: 27 - DNA, 23 bp, Artificial Sequence
  Synthetic construct
gtgacgcaaaataccaccttggc

SEQ ID NO: 28 - DNA, 22 bp, Artificial Sequence
  Synthetic construct
ccattcaccttgcacaccaggg

SEQ ID NO: 29 - DNA, 22 bp, Artificial Sequence
  Synthetic construct
aattgctcctgtccactttgac

SEQ ID NO: 30 - DNA, 23 bp, Artificial Sequence
  Synthetic construct
tcactaactcggaaatccacagt

SEQ ID NO: 31 - DNA, 21 bp, Artificial Sequence
  Synthetic construct
ctggctagtctggtgtactcg

SEQ ID NO: 32 - DNA, 19 bp, Artificial Sequence
  Synthetic construct
atgcccactcgctgattgg

SEQ ID NO: 33 - DNA, 21 bp, Artificial Sequence
  Synthetic construct
gccaatgacacctactggatg

SEQ ID NO: 34 - DNA, 22 bp, Artificial Sequence
  Synthetic construct
gagctgtactctgaccttgttg